(19) United States
(12) Patent Application Publication
(10) Pub. No.: US 2005/0129719 A1
(43) Pub. Date: Jun. 16, 2005

(54) Concentrated emulsion formulation for silatecan

(76) Inventors: Yu-Fang Hu, Taipei (TW); Yao-Kun Huang, Taipei (TW); Huei-Tang Lin, Taipei (TW); Kuo-Hua Yang, Taipei (TW)

Correspondence Address: Jackson Walker L.L.P.;Suite 2100, 112 E. Pecan Street, San Antonio, TX, 78205, US

(21) Appl. No.: 10/736,307

(22) Filed: Dec. 15, 2003

(51) Int. Cl. A61K 31/4745; A61K 9/00; A61K 31/695

(52) U.S. Cl. 424/400; 514/283; 514/63

(57) ABSTRACT

The present application relates to a concentrated emulsion formulation for water-insoluble silatecan comprising silatecan of 0.01-1.0% (W/W), phospholipid of 4.79-75% (W/W); propylene glycol of 24.79˜95% (W/W); optional ethanol of 0.1-40% (W/W); optional surfactant of 0.1-10% (W/W), especially Tween® 80. The present application also relates to a method for manufacturing the concentrated emulsion formulation.

### BACKGROUND OF THE INVENTION

1. Field of the Invention

The present invention relates to a concentrated emulsion formulation, and particularly to a waterless emulsion formulation for water-insoluble silatecan.

2. Description of Related Art

Silatecan (7-t-butyldimethylsilyl-10-hydroxycamptothecin, DB-67) is known to be an inhibitor of topoisomerase, which suppresses tumor cells and also has extremely low water-solubility. Thus, particularly selected solvents must be used for preparation of an injectable silatecan dose. The chemical structure of the silatecan is shown as follows:

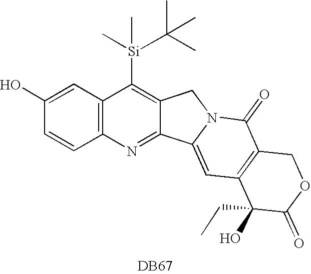

Pharmaceutical compounds are classified into two groups—the water-soluble and the water-insoluble groups. Medicine containing water-soluble compounds is prepared relatively easier. Taking the antitussive “dextromethorphen” as an example, dextromethorphen is easily dissolved in water and thus has been prepared in a variety of pharmaceutical dosage forms, such as tablets, solutions, syrups etc. Another example is the anti-cancer drug “doxorubicin,” which has excellent solubility in water and is easily formulated into an aqueous solution for injection. On the contrary, the manufacturing process of medicine containing water-insoluble effective compounds is usually difficult and complex. For example, the anti-cancer medicine “paclitaxel” underwent a difficult development period before marketing. Then, serious allergic reactions were observed in Phase I of clinical trials of the drug, which almost led to termination of the clinical trials. The allergic reactions included suffocating, hypotension, angioedma, systemic urticaria, all of which were similar to the negative reactions associated with contrasting agents for angiography. By using multiple drugs for prevention and modifying the rate of injection, the allergic reactions caused by paclitaxel were reduced finally. The reason for those allergic reactions was found to be primarily associated with the solvents used in the preparing process of paclitaxel rather than paclitaxel itself.

Paclitaxel is very difficult to dissolve in water (less than 1 μg/mL). Therefore, solvents suitable for preparing a medicament containing paclitaxel must be investigated. This is an obstacle for manufacturing paclitaxel-containing medicine. The only solvent that was disclosed by Squibb U.S. is used for intravenous doses of paclitaxel, composing of ethanol and Cremophor EL (polyoxyethylated castor oil) in a ratio of 50:50. However, Cremophor EL is so toxic that it causes serious allergic reactions, even fatal, after injection.

Obviously, the injectable paclitaxel medicine resulted in the allergic reactions just because of use of Cremophor EL. Therefore, the drawbacks of the paclitaxel-containing medicine can be eliminated by replacing Cremophor EL with any other less toxic solvent. Nonetheless, a suitable solvent for manufacturing paclitaxel-containing medicine should not only render the manipulation of the insoluble drug much easier but also provide sufficient safety as a medicament.

To improve the injection dosage containing paclitaxel compound with low water solubility, the injection dosage was made in the form of liposome. However, the paclitaxel compound in liposome for injection dosage was found to become deposited within several weeks of its shelf life. Therefore, the liposomes were frozen into powder to avoid the deposition of the paclitaxel compound during storage. However, the processes of making the liposomes were complex, and freezing processes are time-consuming with high operational costs, which make the paclitaxel medicine high in production cost and disadvantageous commercially. Other modifications of paclitaxel medicine but having easier manufacturing processes were disclosed. For example, some organic solvents such as dimethylacetamide (DMA) and N-methylpyroolidinone (NMP) were used to substitute the Cremophor EL to increase the solubility of the paclitaxel compound. However, although the aforementioned organic solvents did simplify the manufacturing processes they still had certain toxicity. Therefore, the organic solvents are not widely accepted by pharmaceutical manufacturers.

Another special method was to mix the paclitaxel compound with other non-toxic materials such as using plasma protein to absorb paclitaxel to obtain non-toxic and water-insoluble paclitaxel medicine of an injectable dosage.

Compounds having low water-solubility such as paclitaxel were usually prepared into an emulsion to overcome the solubility problem to make the paclitaxel distributed evenly. Emulsifying the paclitaxel was more complex than using the specific solvents to dissolve the paclitaxel, and thus had a high manufacturing cost. Additionally, the emulsifier used in the emulsifying processes usually contained water that makes the paclitaxel have a poor stability during long-period storage. Meanwhile, the paclitaxel became deposited easily and crystallized in the emulsion form in the same way as in the liposome form.

According to the above description, methods for preparing medicine containing water-insoluble compounds comprised using organic solvents, using surfactant, and using specific carrying media (such as liposome) to achieve the purpose of evenly dispersing the water-insoluble compounds in the medicine in different dosage forms. However, these methods all had problems of excessive toxicity caused from solvents, quality control difficulties due to complex processing etc.

The present invention has arisen to mitigate or obviate the disadvantages of the conventional method for preparing medicine containing water-insoluble compounds, especially silatecan.

### SUMMARY OF THE INVENTION

The first objective of the present invention is to provide an emulsion formulation for silatecan, which contains no toxic excipient and enables other water-insoluble compounds to be applied to reduce side effects of the obtained medicine. Wherein, the objective is achieved by the following emulsion formulation:
phospholipid of 4.79-75% (W/W);
propylene glycol of 24.79˜95% (W/W);
optional ethanol of 0.1-40% (W/W);
optional surfactant of 0.1-10% (W/W), especially Tween® 80; and
silatecan of 0.01-1.0% (W/W).

The second objective of the present invention is to provide an emulsion formulation for silatecan, wherein manufacturing processes of the emulsion formulation are simply to reduce the production cost of the silatecan medicine.

Further benefits and advantages of the present invention will become apparent after a careful reading of the detailed description.

### DETAILED DESCRIPTION OF THE PREFERRED EMBODIMENTS

A concentrated emulsion formulation for silatecan or other water-insoluble compounds, the emulsion formulation comprising:
phospholipid of 4.79-75% (W/W);
propylene glycol of 24.79˜95% (W/W);
optional ethanol of 0.1-40% (W/W);
optional surfactant of 0.1-10% (W/W), especially Tween ®80 and silatecan of 0.01-1.0% (W/W).

The emulsion formulation in the present invention uses non-toxic organic solvents and optional surfactants to dissolve waterless and water-insoluble compounds. The phospholipid is preferred to be phospholipon ®90G, which contains a minimum 90% phosphatidylcholine for the manufacture of liposomes. The phosphatidylcholine is shown in following chemical structure:

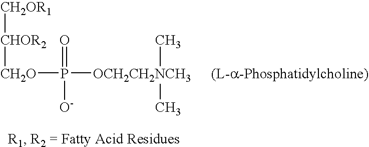

The organic solvents and surfactants are propylene glycol, optional ethanol and optional Tween ®80 (or polysorbate 80, purchased from Fisher Scientific) and are simultaneously mixed with phospholipon ®90G in a mixer without the need for any extra equipment to achieve a pharmaceutically acceptable emulsion. Therefore, the emulsion for silatecan or other water-insoluble compounds has a low manufacturing cost.

Additionally, the emulsion formulation contains no water and thus has an excellent stability such that the emulsion formulation can be preserved for a long time, at least two years at room temperature. Since the emulsion is obtained in a dense situation, the emulsion has to be diluted before administration. Take an intravenous injection for example, the emulsion is diluted with a suitable diluent for the intravenous injection such as an aqueous solution, oily solvent or lipid diluent. The aqueous solution is selected from the group consisting of injection water, glucose solution, saline, Ringer's solution and other injection solutions. The oily solvents are selected from the group consisting of triglycerides, propylene glycol diesters and other mixtures, wherein the triglycerides contain 9-83 carbon atoms and the propylene glycol diesters contain 15-60 carbon atoms. The lipid diluent is selected from the group consisting of commercially-available products such as Liposyn® (purchased from Abbott Laboratories), Soyacal® (purchased from GRIFOLS), Travemulsion®, Intralipid® (purchased from Fresenius Kabi) etc. Wherein, Liposyn® is composed of safflower oil, soybean oil, 1.2% egg phosphatides added as an emulsifier and glycerin in water. Wherein, Soyacal® is a synthetic lipid emulsion, Travemulsion® is a lipid emulsion and Intralipid® is an egg lecithin.

After diluting, the diluted emulsion remains stable and the water-insoluble compound does not decompose or crystallize to a deposit within 24 hours. In fact, if the water-insoluble compounds are sensitive to light, the emulsion provides a photo-resisting efficiency to make the water-insoluble compounds stable and safe.

Silatecan is the main subject studied in the present invention but other water-insoluble compounds are also applicable in the emulsion formulation. Without intending to limit in any manner, the present invention is further illustrated by the following examples.

### EXAMPLE 1

### Concentrated Emulsion Formulation Containing 3 mg/mL Silatecan

Ingredient of the emulsion formulation is listed on table 1.

TABLE 1
ingredient of 3 mg/mL silatecan concentrated emulsion

| Compound | Quantity | |
|---|---|---|
| Silatecan (DB67) | 0.31 | g |
| Phospholipon 90G | 34.9 | g |
| Propylene glycol | 52.9 | g |
| Ethanol q.s.(quantum sufficit) | 14.0 | mL |

Silatecan, phospholipon, propylene glycol, and ethanol were simultaneously mixed together, heated in a water-bath at 70-80° C., and then stirred by a magnet for 2 hours to obtain 3 mg/mL waterless emulsion of silatecan. The silatecan emulsion was divided into 4 parts to be individually stored at 5° C.±2° C., 25° C.±2° C., 30° C.±2° C., 40° C.±2° C. All parts of the silatecan emulsion were tested for concentration variation and had no obvious deposition or decomposition within 6 months, as shown in Table 2.

TABLE 2
stability test for 3 mg/mL silatecan concentrated emulsion

| Storage | Concentration mg/mL | | | |
|---|---|---|---|---|
| period | 5° C. ± 2° C. | 25° C. ± 2° C. | 30° C. ± 2° C. | 40° C. ± 2° C. |
| 0 | 3.04 ± 0.18 | 3.04 ± 0.18 | 3.04 ± 0.18 | 3.04 ± 0.18 |
| 1^{st} month | 3.11 ± 0.22 | 3.12 ± 0.13 | 3.18 ± 0.21 | 3.10 ± 024 |
| 2^{nd} month | 2.98 ± 0.11 | 3.02 ± 0.17 | 3.05 ± 0.19 | 3.12 ± 0.19 |
| 3^{rd} month | 3.12 ± 0.28 | 2.09 ± 0.18 | 3.12 ± 0.22 | 3.08 ± 0.26 |
| 6^{th} month | 2.97 ± 0.15 | 3.18 ± 0.22 | 2.95 ± 0.23 | 3.11 ± 0.15 |

A 3.5 mL sample of 3 mg/mL silatecan concentrated emulsion was diluted with 100 mL of 5% dextrose solution or intravenous injecting water and stirred for 1 minute to obtain a homogeneous liquid solution. To study the stability and crystallization of the silatecan concentrated emulsion after diluting, the liquid solution was filtered with a membrane of 0.45 μm pores and then the filtered liquid solution was tested for concentration variation to determine the crystallizing ratio. The stability test was held at the beginning, 4^{th} hour, 8^{th} hour, 12^{th} hour, and 24^{th} hour after diluting (shown in Table 3).

TABLE 3
stability test for diluted silatecan emulsion after diluting.

Concentration mg/mL*

| Storage period | Before filtering | After filtering |
|---|---|---|
| 0 | 0.11 ± 0.01 | 0.10 ± 0.02 |
| 4^{th} hour | 0.13 ± 0.01 | 0.11 ± 0.03 |
| 8^{th} hour | 0.11 ± 0.02 | 0.10 ± 0.03 |
| 12^{th} hour | 0.10 ± 0.02 | 0.12 ± 0.01 |
| 24^{th} hour | 0.12 ± 0.01 | 0.13 ± 0.02 |

*tested with HPLC

### EXAMPLE 2

### Concentrated Emulsion Formulation Containing 6 mg/mL Silatecan

The ingredients of the emulsion formulation are listed in Table 4, which further contain 1% Tween ®80 to diminish the amount of droplets of the emulsion to half the size of the droplets in example 1 and to condense the silatecan concentrated emulsion to 6 mg/mL.

TABLE 4
ingredient of 6 mg/mL silatecan concentrated emulsion

| Compound | Quantity | |
|---|---|---|
| Silatecan (DB67) | 0.61 | g |
| Phospholipon 90G | 34.5 | g |
| Propylene glycol | 52.4 | g |
| Tween ® 80 | 1.0 | mL |
| Ethanol q.s.(quantum sufficit) | 14.0 | mL |

Silatecan, phospholipon, propylene glycol, Tween ®80 and ethanol were simultaneously mixed together, heated in a water-bath at 70-80° C., and then stirred by a magnet for 2 hours to obtain 6 mg/mL waterless emulsion of silatecan. The silatecan emulsion was divided into 4 parts to be individually stored at 5° C.±2° C., 25° C.±2° C., 30° C.±2° C., 40° C.±2° C. All parts of the silatecan emulsion were tested for concentration variation and had no obvious deposition or decomposition within 6 months as shown in Table 5.

TABLE 5
stability test for 6 mg/mL silatecan concentrated emulsion

| Storage | Concentration mg/mL | | | |
|---|---|---|---|---|
| period | 5° C. ± 2° C. | 25° C. ± 2° C. | 30° C. ± 2° C. | 40° C. ± 2° C. |
| 0 | 6.01 ± 0.20 | 6.01 ± 0.20 | 6.01 ± 0.20 | 6.01 ± 0.20 |
| 1^{st} month | 6.04 ± 0.23 | 5.97 ± 0.15 | 6.15 ± 0.11 | 6.04 ± 0.21 |
| 2^{nd} month | 6.12 ± 0.18 | 6.15 ± 0.19 | 5.91 ± 0.18 | 6.03 ± 0.20 |
| 3^{rd} month | 5.97 ± 0.22 | 5.96 ± 0.22 | 6.11 ± 0.14 | 5.91 ± 0.11 |
| 6^{th} month | 6.17 ± 0.23 | 6.05 ± 0.14 | 5.90 ± 0.21 | 6.01 ± 0.14 |

A 9.0 mL sample of 6 mg/mL silatecan concentrated emulsion was diluted with 100 mL of 5% dextrose solution or intravenous injecting water and stirred for 1 minute to obtain a homogeneous liquid solution. To study the stability and crystallization of the silatecan concentrated emulsion after diluting, the liquid solution was filtered with a membrane of 0.45 μm pores and then the filtered liquid solution was tested for concentration variation to determine the crystallizing ratio. The stability test was held at the beginning, 4^{th} hour, 8^{th} hour, 12^{th} hour, and 24^{th} hour after diluting (shown in Table 6).

TABLE 6
stability test for diluted silatecan emulsion after diluting.

Concentration mg/mL*

| Storage period | Before filtering | After filtering |
|---|---|---|
| 0 | 0.50 ± 0.03 | 0.49 ± 0.01 |
| 4^{th} hour | 0.49 ± 0.02 | 0.52 ± 0.02 |
| 8^{th} hour | 0.55 ± 0.02 | 0.52 ± 0.03 |
| 12^{th} hour | 0.47 ± 0.02 | 0.49 ± 0.04 |
| 24^{th} hour | 0.50 ± 0.03 | 0.52 ± 0.02 |

*tested with HPLC

The concentrated silatecan emulsion in the present invention was simply obtained by mixing multiple non-toxic solvents and a surfactant together to simplify the manufacturing process of water-insoluble compounds. Therefore, the concentrated silatecan emulsion is non-toxic and has a low manufacturing cost. Meanwhile, the concentrated silatecan emulsion is waterless to keep the silatecan stable in the emulsion and being waterless even keeps it stable for at least 24 hours after diluting.

Although the invention has been explained in relation to its preferred embodiment, many other possible modifications and variations can be made without departing from the spirit and scope of the invention as hereinafter claimed.

1. A concentrated emulsion formulation for silatecan comprising:
phospholipid of 4.79-75% (W/W);
propylene glycol of 24.79˜95% (W/W); and
silatecan of 0.01-1.0% (W/W).

2. The concentrated emulsion formulation for silatecan as claimed in claim 1, which further comprises ethanol of 0.1-40% (W/W).

3. The concentrated emulsion formulation for silatecan as claimed in claim 1, which further comprises Tween®80 of 0.1-10% (W/W).

4. The concentrated emulsion formulation for silatecan as claimed in claim 1, wherein the concentrated emulsion formulation is diluted with a diluent before administration.

5. The concentrated emulsion formulation for silatecan as claimed in claim 4, wherein the diluent is selected from the group consisting of injection water, dextrose solution, saline and Ringer's solution.

6. The concentrated emulsion formulation for silatecan as claimed in claim 4, wherein the diluent is selected from the group consisting of triglyceride, propylene glycol diester and a mixture thereof.

7. The concentrated emulsion formulation for silatecan as claimed in claim 6, wherein the triglyceride contains 9-83 carbon atoms.

8. The concentrated emulsion formulation for silatecan as claimed in claim 6, wherein the propylene glycol diester contains 15-60 carbon atoms.

9. The concentrated emulsion formulation for silatecan as claimed in claim 4, wherein the diluent is selected from the group consisting of Liposyn®, Soyacal®, Travemulsion® and Intralipid®.

10. A method for manufacturing a concentrated emulsion formulation for silatecan as claimed in claim 1, comprising:
simultaneously mixing 0.01-1.0% (W/W) silatecan together with 4.79-75% (W/W) phospholipid and 24.79˜95% (W/W) propylene glycol to form a mixture; and
stirring the mixture evenly until the mixture is completely emulsified.

11. The method as claimed in claim 10, which further comprises adding 0.1-40% (W/W) ethanol to the mixture.

12. The method as claimed in claim 10, which further comprises adding 0.1-10% (W/W) Tween®80 to the mixture.
